(19) United States
(12) Patent Application Publication
(10) Pub. No.: US 2013/0116326 A1
(43) Pub. Date: May 9, 2013

(54) TREATMENT OF DELAYED CUTANEOUS HYPERSENSITIVITY CONDITIONS WITH S-FARNESYLTHIOSALICYLIC ACID AND ANALOGS THEREOF

(71) Applicants: Yoseph A. Mekori, Kfar Saba (IL); Adam Mor, Tel-Aviv (IL); Yoel Kloog, Herzliya (IL)

(72) Inventors: Yoseph A. Mekori, Kfar Saba (IL); Adam Mor, Tel-Aviv (IL); Yoel Kloog, Herzliya (IL)

(73) Assignee: RAMOT AT TEL-AVIV UNIVERSITY LTD., Tel Aviv (IL)

(21) Appl. No.: 13/809,000

(22) PCT Filed: Jul. 7, 2011
(86) PCT No.: PCT/IL2011/000540

§ 371 Date: Jan. 8, 2013

Related U.S. Application Data
(60) Provisional application No. 61/362,496, filed on Jul. 8, 2010.

(51) Int. Cl. A61K 31/192; A61K 31/165

(52) U.S. Cl. 514/568

(57) ABSTRACT

Disclosed are methods of treating a mammalian subject afflicted with a delayed cutaneous hypersensitivity condition, comprising administering to the subject a pharmaceutical composition comprising an effective amount of S-farnesylthiosalicylic acid (FTS) or a structural analog thereof, and compositions for use in the methods.

### CROSS-REFERENCE TO RELATED APPLICATIONS

The present application is a national phase entry under 35 U.S.C. §371 of International Application No. PCT/IL2011/000540, filed Jul. 7, 2011, published in English, which claims priority from U.S. Provisional Application No. 61/362,496, filed Jul. 8, 2010, all of which are incorporated herein by reference.

### BACKGROUND OF THE INVENTION

Classic delayed-type hypersensitivity and allergic contact dermatitis are common clinical problems. Among the many contact sensitizing antigens to which humans are exposed include drugs, dyes, plant oleo resins, preservatives, and metals. Five common contact sensitizing agents encountered in clinical practice are Rhus species of plants (poison ivy, oak, or sumac), paraphenylenediamine, nickel compounds, rubber compounds, and the dichromates. They can lead to delayed hypersensitivity responses, which may represent significant medical problems.

Delayed (or delayed-type) cutaneous hypersensitivity is a T-cell dependent immune phenomenon manifested by an inflammatory reaction of the skin, at the site of antigen deposition, that typically reaches its peak intensity 24 to 48 hours after challenge by the antigen. It is quite common in humans, having a median prevalence of 21%. See, Thyssen, et al., Contact Dermatitis 57:287-99 (2007); Carlsen, et al., Contact Dermatitis 58:1-8 (2008). This phenomenon is often the result of exposure to contact sensitizing antigens (also known as contact sensitivity or contact dermatitis). Contact dermatitis includes irritant dermatitis, phototoxic dermatitis, allergic dermatitis or allergic contact dermatitis, photoallergic dermatitis, contact urticaria, systemic contact-type dermatitis and the like.

The simplest treatment of allergic contact dermatitis is avoidance of exposure to an identified allergen, but avoiding known allergens may prove difficult. For example, common sensitizers such as benzocaine are employed in a variety of topical medications such as sunburn preparations and antiseptic creams. Unwitting exposure to a known allergen such as poison ivy can occur through contact with the smoke of burning leaves. In addition, the patient may exacerbate an allergic contact dermatitis by exposure to cross-reacting chemical compounds that are similar to the allergen by which the patient was originally sensitized.

Symptomatic treatment usually involves the application of topical corticosteroids. Prolonged topical use of corticosteroids can produce undesirable side effects such as atrophy of the skin, systemic absorption of the corticosteroids, and reduced immune defense resulting in a secondary bacterial infection, particularly of fungi such as Candida. Further, such treatment requires frequent suspension of the treatment, and such treatment cannot be used during the exudative acute phase of the dermatitis. Oral or parenteral corticosteroids may be needed temporarily in severe cases, but long term therapy with exogenous corticosteroids are associated with undesired effects such as Cushing's syndrome, adrenal insufficiency, osteoporosis, secondary diabetes, hypertension, and cataract formation.

### BRIEF SUMMARY OF THE INVENTION

According to a first aspect, the present invention is directed to the compound S-farnesylthiosalicylic acid (FTS) or a structural analog thereof, collectively defined in accordance with formula (I) herein for use in treating a mammalian subject afflicted with a delayed cutaneous hypersensitivity condition.

Another aspect of the present invention is directed to a method of treating a mammalian subject afflicted with a delayed cutaneous hypersensitivity condition, comprising administering to the subject a pharmaceutical composition comprising an effective amount of S-farnesylthiosalicylic acid (FTS) or a structural analog thereof, collectively defined in accordance with formula (I) herein, and a pharmaceutically acceptable carrier. The indications treatable in accordance with the present invention are non-cancerous and non-autoimmune in nature.

Applicants have surprisingly and unexpectedly found that FTS inhibits the activation phase of delayed cutaneous hypersensitivity in vivo, and that this effect is associated with inhibition of Rap1 more than with the inhibition of Ras (which FTS is known to inhibit). Accordingly, a related aspect of the present invention is directed to a method of inhibiting Rap1 in vivo, comprising administering to a mammalian subject afflicted with a delayed cutaneous hypersensitivity condition a pharmaceutical composition comprising an effective amount of S-farnesylthiosalicylic acid (FTS) or a structural analog thereof, collectively defined in accordance with formula (I) herein, and a pharmaceutically acceptable carrier.

In a further aspect, the invention relates to a composition comprising S-farnesylthiosalicylic acid (FTS) or a structural analog thereof, collectively defined in accordance with formula (I) herein as an active agent for treating a delayed cutaneous hypersensitivity condition.

Another aspect of the invention relates to the use of S-farnesylthiosalicylic acid (FTS) or a structural analog thereof, collectively defined in accordance with formula (I) herein for the preparation of a medicament for treating a delayed cutaneous hypersensitivity condition.

In yet another aspect, the invention is directed to the use of S-farnesylthiosalicylic acid (FTS) or a structural analog thereof, as collectively defined above for treating a mammalian subject afflicted with a delayed cutaneous hypersensitivity condition.

In some embodiments of all aspects of the invention, FTS-amide (FTS-A) is used for treatment, for preparation of a pharmaceutical composition or is the active agent in a composition for treating a delayed cutaneous hypersensitivity condition.

### BRIEF DESCRIPTION OF THE DRAWINGS

FIGS. 1A-C show that FTS analogs inhibit Rap1 in Jurkat T cells.

FIG. 2 shows that FTS analogs inhibit T cell adhesion.

FIGS. 3A-C show that FTS-A inhibition of Rap1 is dose-dependent.

FIGS. 4A-B show that FTS-A inhibits Rap1 activation at the plasma membrane.

FIGS. 5A-B show that FTS-A inhibits contact sensitivity reaction in animal model.

FIGS. 6A-B show that Rap1 inhibition by FTS-A is not associated with Ras inhibition.

### DETAILED DESCRIPTION

In some embodiment of the present invention, the delayed cutaneous hypersensitivity condition treated by the methods and pharmaceutical compositions of the invention, is contact dermatitis, which as known in the art, includes conditions such as irritant dermatitis, phototoxic dermatitis, allergic dermatitis or allergic contact dermatitis, photoallergic dermatitis, contact urticaria, systemic contact-type dermatitis and the like. Common signs or symptoms of contact dermatitis, any one or more of which the methods of the present invention may alleviate, include redness, pain, itching and swelling. Sometimes blistering and weeping of the skin also develop. The clinical symptoms of contact dermatitis can include acute eczema accompanied by erythema, edema, papula, vesicle, erosion, and itching. Repeated exposure to an irritant can lead to the development of eczema accompanying lichenification and infiltration.

Allergic contact dermatitis can appear after initial or prolonged exposure to an irritant. A wide range of agents can cause allergic contact dermatitis including for example, Rhus species of plants (poison ivy, oak, and sumac), metals (e.g., nickel, chromium, cobalt), fragrances, chemicals, cosmetics, textiles, pesticides, plastics (e.g., latex), and pollen (see, for example, R. J. G. Rycroft, et al. “Textbook of Contact Dermatitis”). Therapeutic agents such as drugs may also cause allergic contact dermatitis, particularly (but not exclusively) when administered transdermally. It is well known that many drugs, e.g., topical ointments, including some currently marketed in the United States (e.g., clonidine) sensitize the skin when used.

Irritant dermatitis can occur when too much of a substance is used on the skin or when the skin is sensitive to a certain substance. Susceptibility can include a genetic component. Skin-irritating agents are substances (e.g., soap) that cause an immediate and generally localized adverse response. The response is typically in the form of redness and/or inflammation and generally does not extend beyond the immediate area of contact. Symptoms that are commonly seen include redness, scaling, and the skin looking irritated and sore.

In another embodiment of the present invention, the delayed cutaneous hypersensitivity condition is atopic (endogenous) dermatitis, sometimes referred to as eczema. This condition is caused by exposure to various antigens, since an individual has an atopic disposition which is hypersensitivity against a certain substance. The clinical symptoms include marked itching, skin hypertrophy, infiltration, lichenification and the like.

The subjects for treatment with the methods and pharmaceutical compositions of the present invention are mammals, including humans and experimental or disease-model mammals, and other non-human mammals including domestic animals.

FTS and its structural analogs useful in the methods, uses and pharmaceutical compositions of the present invention may be collectively represented by the following

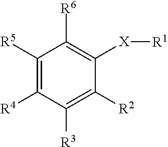

formula:
wherein

X represents S;

wherein R¹ represents farnesyl or geranyl-geranyl;

R² is COOR^{7,} CONR⁷R⁸, or COOCHR⁹OR¹⁰, wherein R⁷ and R⁸ are each independently hydrogen, alkyl, or alkenyl, including linear and branched alkyl or alkenyl, which in some embodiments includes C1-C4 alkyl or alkenyl;

wherein R⁹ represents H or alkyl; and

wherein R¹⁰ represents alkyl, including linear and branched alkyl and which in some embodiments represents C1-C4 alkyl; and

wherein R³, R⁴, R⁵ and R⁶ are each independently hydrogen, alkyl, alkenyl, alkoxy (including linear and branched alkyl, alkenyl or alkoxy and which in some embodiments represents C1-C4 alkyl, alkenyl or alkoxy), halo, trifluoromethyl, trifluoromethoxy, or alkylmercapto.

In embodiments wherein any of R⁷, R⁸, R⁹ and R¹⁰ represents alkyl, it is methyl or ethyl.

Thus, aside from FTS (e.g., the isomer S-trans,trans-farnesylthiosalicylic acid, wherein R¹ is farnesyl, R² is COOR⁷, and R⁷ is hydrogen), in some embodiments, the FTS analog is halogenated, e.g., 5-chloro-FTS (wherein R¹ is farnesyl, R² is COOR⁷, R⁴ is chloro, and R⁷ is hydrogen), and 5-fluoro-FTS (wherein R¹ is farnesyl, R² is COOR⁷, R⁴ is fluoro, and R⁷ is hydrogen).

In other embodiments, the FTS analog is FTS-methyl ester (wherein R¹ represents farnesyl, R² represents COOR⁷, and R⁷ represents methyl).

In yet other embodiments, the FTS analog is an alkoxyalkyl S-prenylthiosalicylate or an FTS-alkoxyalkyl ester (wherein R² represents)COOCHR⁹OR¹⁰. Representative examples include methoxymethyl S-farnesylthiosalicylate (wherein R¹ is farnesyl, R⁹ is H, and R¹⁰ is methyl); methoxymethyl S-geranylgeranylthiosalicylate (wherein R^{1l} is geranylgeranyl, R⁹ is H, and R¹⁰ is methyl); methoxymethyl 5-fluoro-S-farnesylthiosalicylate (wherein R¹ is farnesyl, R⁵ is fluoro, R⁹ is H, and R¹⁰ is methyl); and ethoxymethyl S-farnesylthiosalicyate (wherein R¹ is farnesyl, R⁹ is methyl and R¹⁰ is ethyl). In each of the embodiments described above, unless otherwise specifically indicated, each of R³, R⁴, R⁵ and R⁶ represents hydrogen.

In some embodiments, the FTS analog is FTS-amide (wherein R¹ represents farnesyl, R² represents CONR⁷R⁸, and R⁷ and R⁸ both represent hydrogen); FTS-methylamide (wherein R¹ represents farnesyl, R² represents CONR⁷R⁸, R⁷ represents hydrogen and R⁸ represents methyl); or FTS-dimethylamide (wherein R¹ represents farnesyl, R² represents CONR⁷R⁸, and R⁷ and R⁸ each represents methyl).

The term “alkyl” refers to a saturated aliphatic hydrocarbon having between 1 and 12 carbon atoms, in some embodiments between 1 and 6 carbon atoms, which may be arranged as a straight chain or branched chain, or as a cyclic group. These are, for example, methyl, ethyl, propyl, isobutyl, and butyl.

The alkyl group may be unsubstituted or substituted with one or more of a variety of groups selected from halogen, hydroxyl, alkyloxy, alkylthio, arylthio, alkoxy, alkylcarbonyl, carbonyl, alkoxycarbonyl, ester, amido, alkylamido, dialkylamido, aryl, benzyl, aryloxy, nitro, amino, alkyl or dialkylamino, carboxyl, thio, and others, each optionally being isotopically labeled. When substituted by a terminal group, the alkyl is an alkylene having between 1 and 12 carbon atoms. When the alkyl or alkylene group contains one or more double bonds it is referred herein as an “alkenyl”.

The term “alkoxy” as used herein refers to the —O-(alkyl) group, where the point of attachment is through the oxygen-atom and the alkyl group is as defined hereinbefore.

The term “halogen” or “halo” as used herein refers to —Cl, —Br, —F, or —I groups.

The term “ester” as used herein refers to a —C═(O)—O—, where the points of attachment are through both the C-atom and O-atom. One or both oxygen atoms of the ester group can be replaced with a sulfur atom, thereby forming a “thioester”, i.e., a —C═(O)—S—, —C═(S)—O— or —C═(S)—S— group.

The term “about” refers herein to 10% more or less of the value which it refers to.

### Compositions and Methods

The term “treatment” as used herein refers to the administering of a therapeutic amount of the composition of the present invention which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease form occurring or a combination of two or more of the above.

The term “effective amount” as used herein, refers to a sufficient amount of an active ingredient as represented by formula (I) that will ameliorate at least one symptom or underlying biochemical manifestation of a delayed cutaneous hypersensitivity condition, such as inhibition of Rap1, for example, diminish extent or severity or delay or retard progression, or achieve complete healing and regression of the condition. Appropriate “effective” amounts for any subject can be determined using techniques, such as a dose escalation study. Specific dose levels for any particular subject will depend on several factors such as the potency of the active ingredient represented by formula (I), the age, weight, and general health of the subject, and the severity of the disorder. The average daily dose of the active ingredient of formula (I) generally ranges from about 200 mg to about 2000 mg, in some embodiments from about 400 to about 1600 mg, and some other embodiments from about 600 to about 1200 mg, and in yet other embodiments, from about 800 mg to about 1200 mg.

The terms “administer,” “administering”, “administration,” and the like, as used herein, refer to the methods that may be used to enable delivery of the active ingredient to the desired site of biological action. Medically acceptable administration techniques suitable for use in the present invention are known in the art. See, e.g., Goodman and Gilman, The Pharmacological Basis of Therapeutics, current ed.; Pergamon; and Remington's, Pharmaceutical Sciences (current edition), Mack Publishing Co., Easton, Pa. In some embodiments, the active ingredient is administered orally. In other embodiments, the active ingredient is administered parenterally (which for purposes of the present invention, includes intravenous, subcutaneous, intraperitoneal, intramuscular, intravascular and infusion). In yet other embodiments, the active ingredient is administered transdermally (e.g., topically). As used herein, topical administration refers to non-enteral and non-parenteral modes of administration, and thus includes direct or indirect application to the skin, as well as inhalational (e.g., via aerosol) and ocular (e.g., eye drops or eardrops) administration.

The term “pharmaceutical composition,” as used herein, refers to a combination or mixture of the active ingredient and a pharmaceutically acceptable carrier, and optionally a pharmaceutically acceptable excipient, which as known in the art include substances or ingredients that are non-toxic, physiologically inert and do not adversely interact with the active ingredient of formula (I) (and any other additional active agent(s) that may be present in the composition). Carriers facilitate formulation and/or administration of the active agents.

The term “pharmaceutically acceptable carrier” (which is interchangeably referred to throughout the specification as “carriers”) refers to any vehicle, adjuvant, excipient, diluent, which is known in the field of pharmacology for administration to a human subject and is approved for such administration. The choice of carrier will be determined by the particular active agent, for example, its dissolution in that specific carrier (hydrophilic or hydrophobic), as well as by other criteria such as the mode of administration.

Oral compositions suitable for use in the present invention may be prepared by bringing the active ingredient(s) into association with (e.g., mixing with) the carrier, the selection of which is based on the mode of administration. Carriers are generally solid or liquid. In some cases, compositions may contain solid and liquid carriers. Compositions suitable for oral administration that contain the active are, according to some embodiments of the invention, in solid dosage forms such as tablets (e.g., including film-coated, sugar-coated, controlled or sustained release), capsules, e.g., hard gelatin capsules (including controlled or sustained release) and soft gelatin capsules, powders and granules. The compositions, however, may be contained in other carriers that enable administration to a patient in other oral forms, e.g., a liquid or gel. Regardless of the form, the composition is divided into individual or combined doses containing predetermined quantities of the active ingredient.

Oral dosage forms may be prepared by mixing the active ingredient, typically in the form of an active pharmaceutical ingredient with one or more appropriate carriers (optionally with one or more other pharmaceutically acceptable excipients), and then formulating the composition into the desired dosage form e.g., compressing the composition into a tablet or filling the composition into a capsule (e.g., a hard of soft gelatin capsule) or a pouch. Typical carriers and excipients include bulking agents or diluents, binders, buffers or pH adjusting agents, disintegrants (including crosslinked and super disintegrants such as croscarmellose), glidants, and/or lubricants, including lactose, starch, mannitol, microcrystalline cellulose, ethylcellulose, sodium carboxymethylcellulose, hydroxypropylmethylcellulose, dibasic calcium phosphate, acacia, gelatin, stearic acid, magnesium stearate, corn oil, vegetable oils, and polyethylene glycols. Coating agents such as sugar, shellac, and synthetic polymers may be employed, as well as colorants and preservatives. See, Remington's Pharmaceutical Sciences, The Science and Practice of Pharmacy, 20th Edition, (2000).

Liquid form compositions include, for example, solutions, suspensions, emulsions, syrups, elixirs and pressurized compositions. The active agent(s), for example, can be dissolved or suspended in a pharmaceutically acceptable liquid carrier such as water, an organic solvent (and mixtures thereof), and/or pharmaceutically acceptable oils or fats. Examples of liquid carriers for oral administration include water (particularly containing additives as above, e.g., cellulose derivatives, according to some embodiments of the invention, in suspension in sodium carboxymethyl cellulose solution), alcohols (including monohydric alcohols (including monohydric alcohols and polyhydric alcohols, e.g., glycerin and non-toxic glycols) and their derivatives, and oils (e.g., fractionated coconut oil and arachis oil). The liquid composition can contain other suitable pharmaceutical excipients such as solubilizers, emulsifiers, buffers, preservatives, sweeteners, flavoring agents, suspending agents, thickening agents, colorants, viscosity regulators, stabilizers and osmoregulators.

Carriers suitable for preparation of compositions for parenteral administration include Sterile Water for Injection, Bacteriostatic Water for Injection, Sodium Chloride Injection (0.45%, 0.9%), Dextrose Injection (2.5%, 5%, 10%), Lactated Ringer's Injection, and the like. Dispersions can also be prepared in glycerol, liquid polyethylene glycols and mixtures thereof, and in oils. Compositions may also contain tonicity agents (e.g., sodium chloride and mannitol), antioxidants (e.g., sodium bisulfite, sodium metabisulfite and ascorbic acid) and preservatives (e.g., benzyl alcohol, methyl paraben, propyl paraben and combinations of methyl and propyl parabens).

Transdermal (e.g., topical) compositions may take a variety of forms such as gels, creams, lotions, aerosols and emulsions. Representative carriers thus include lubricants, wetting agents, emulsifying and suspending agents, preservatives, anti-irritants, emulsion stabilizers, film formers, gel formers, odor masking agents, resins, hydrocolloids, solvents, solubilizers, neutralizing agents, permeation accelerators, pigments, quaternary ammonium compounds, refatting and superfatting agents, ointment, cream or oil base materials, silicone derivatives, stabilizers, sterilizing agents, propellants, drying agents, opacifiers, thickeners, waxes, emollients, and white oils In addition, the topical preparations of the present invention can be applied and then covered with a bandage, or patch, or some other occlusive barrier, or may be provided as part of a pre-made, ready-to-use topical device, such as a bandage, pad, patch (e.g., transdermal patch of the matrix or reservoir type) or the like. Thus, the composition containing the active ingredient of formula (I) may be applied to a gauze, pad, swab, cotton ball, batting, bandage, patch or occlusive barrier, or in a well or reservoir or as part of a unitary adhesive or nonadhesive mixture, or sandwiched between a peelable or removable layer and a backing layer, which often forms the reservoir, which is occlusive.

Carriers for aerosol formulation, in which the active may be present in finely divided or micronized form, include lactose and propellants such as hydrocarbons (SCF) (propane and n-butane), ether-based poropeilants such as dimethyi ether and methyl ethyl ether, and hydrofluoroalkanes (HFC) such as HFA 134a and HFA 227. Excipients may also be present, e.g., for such purposes as to improve drug delivery, shelf life and patient acceptance. Examples of excipients include wetting agents (e,g., surfactants), dispersing agents, coloring agents, taste masking agents, buffers, antioxidants and chemical stabilizers.

The compound FTS or an analogue thereof of formula (I) may be used as an active ingredient alone or in conjunction with other anti-inflammatory agents such as glucocorticosteroids (e.g., hydrocortisone, prednisone, prednisolone, dexamethasone, betamethasone) and non steroidal anti-inflammatory drugs (e.g., ibuprofen, naproxen, ketoprofen, diclofenac, piroxicam, celecoxib, and etoricoxib).

The pharmaceutical composition containing the active ingredient of formula (I), and optionally another anti-inflammatory agent, may be packaged and sold in the form of a kit. For example, the composition might be in the form of one or more oral dosage forms such as tablets or capsules. The kit may also contain written instructions for carrying out the inventive methods as described herein.

In general, treatment regimens may be designed and optimized by those skilled in the art. For example, the active may be administered until demonstrable symptoms of the inflammatory condition have substantially diminished or the condition is substantially alleviated or healed.

### EXAMPLES

The present invention will now be described in terms of the following non-limiting working examples.

### Introduction

Lymphocytes are the major cellular component of the adaptive immune response. Normal function of lymphocytes depends on several small guanosine nucleotide-binding proteins (small G proteins). This super-family of proteins consists of over 50 members that cycle between an inactive GDP-bound state and an active GTP-bound state. These proteins are involved in a variety of signal transduction pathways that regulate lymphocyte growth, trafficking, migration, and apoptosis (1). Among the most studied are Ras, Rheb, Rho, Rac, and Rap1 (1). Rap1h is highly expressed in T lymphocytes and is related to Ras since the effector domain sequences of the two small G proteins are identical. Interestingly, Rap1 was first identified as an antagonist for Ras. Active Rap1 can bind but not activate Raf-1, which is a downstream effector of Ras. Ras activates Raf-1 that carries its activation signal downstream in the signaling pathway, such that Rap1 may sequester Raf-1 from the Ras/ERK (extracellular signal-regulated kinase) pathway (2). This aspect of Rap1 signaling has been proposed to mediate some of the anti-proliferative actions of Rap1 and underlines the role of Rap1 in anergy, a state in which lymphocytes fail to respond to a specific antigen. T cell receptor (TCR) activation of Rap1 is inhibited by CD28 and is enhanced by CTLA4. This finding is a leading example of the Rap1-Ras complexity (2), since other TCR downstream effectors, including Ras, are enhanced by CD28 and inhibited by CTLA4. The mechanism by which CD28 regulates Rap1 activity is through Rap1-GTPase-activating protein (GAP) induction.

Another pathway through which Rap1 can antagonize Ras function in T cells is the suppression of Ras-dependent reactive oxygen species. Finally, p38 activation by interleukin (IL)-1, characterized as Ras dependent, is antagonized by Rap1. Although the concept of Ras antagonism by Rap1 remains with somewhat controversial, this functional antagonism operates in lymphocytes (3).

Growth control, protein trafficking and polarity are some of the processes in which Rap1 has been implicated. It is believed to be critical with respect to lymphocyte adhesion and migration. The best-characterized and most prominent function of Rap1 is to promote lymphocyte function associated antigen (LFA)-1-mediated adhesion. LFA-1 activation by Rap1 is a critical step for lymphocytes homing to peripheral lymph nodes and migrating into inflamed tissues (3). Inhibition of Rap1 abrogates LFA-1-mediated adhesion to antigen presenting cells (APC) and IL-2 production (2). In some patients with leukocyte adhesion deficiency (LAD) III, a defect in Rap1 GTP loading is responsible for the profound defect in lymphocyte adhesiveness (4, 5), highlighting the critical role of Rap1 in host defense.

Surprisingly, Applicants have found that FTS inhibits the activation phase of delayed cutaneous hypersensitivity in vivo, and that this effect was associated with inhibition of Rap1 more than with the inhibition of Ras. The results of experiments described below also demonstrated that FTS-amide was more potent than FTS in terms of inhibition of Rap1 and contact sensitivity.

### Materials and Methods

### General Reagents

The compound 5-carboxyfluorescein and Opti-MEMI were purchased from Invitrogen Corporation/Molecular Probes (Carlsbad, Calif.). Bryostatin-1 was provided by Sigma-Aldrich (St. Louis, Mo.). FTS was synthesized as previously described (8) and was stored in chloroform, which was evaporated under a stream of nitrogen immediately before use.

FTS-methoxymethylester (FTS-MOM) and FTS-Amide (FTS-A) were provided by Concordia Pharmaceuticals Inc., (Fort Lauderdale, Fla.).

### Antibodies and DNA Constructs

Mouse anti-human CD3 (Ancell, Bayport, MN) was used for T cell activation. Anti-Rap1 antibody was purchased from BD Biosciences (San Jose, Calif.). Monoclonal anti-Ras antibody (Ras10) was purchased from Calbiochem (San Diego, Calif.). GFP-Rap1WT, pcDNA-Rap1WT, pcDNA-Rap1N17, GFP-RalGDS_{RBD}, shRNA-RFP/H1-PLD1, shRNA-Scramble, shRNA-N-Ras, and shRNA-K-Ras constructs were described and validated previously (4, 6).

### Cell Culture, Transfection, and Stimulation

Jurkat T cells were obtained from the American Type Culture Collection (ATCC) (Manassas, VA). The cells were maintained in RPMI 1640 supplemented with 10% FCS, 2 mM 1-glutamine, and 1% penicillin/streptomycin (Biological Industries, Kibutz Beit Haemek, Israel). Panc-1 cells (ATCC) were grown in Dulbecco's modified Eagle medium (DMEM) containing 10% FCS, 2 mM 1-glutamine, and 1% penicillin/streptomycin. The cells were incubated at 37° C. in a humidified atmosphere of 95% air and 5% CO₂. Transfection of Jurkat cells was performed with DMRIE-C (Invitrogen, Carlsbad, Calif.), and cells were examined 24 to 48 hours later. Jurkat T cells were serum starved at 37° C. for 2 to 6 hours, followed by stimulation with 5 μg/ml of mouse anti-human CD3.

### GST-Pull Down Assay

Detection of activated Ras and Rap1 was performed as described previously (4, 9).

### SDS-PAGE and Immunoblotting

Samples were separated by SDS-PAGE using 10% polyacrylamide gels and transferred to nitrocellulose filters. Blots were blocked for 1 hour in TBST (10 mM Tris-HCl (pH 8.0), 150 mM NaCl, and 0.05% Tween 20) containing 3% serum bovine albumin, followed by overnight incubation at 4° C. with the primary antibodies. Blots were washed and incubated for 1 hour at room temperature with HRP-conjugated secondary antibodies. Immunoreactive bands were visualized using the LAS-3000 imaging system (Fujifilm Corp., Tokyo).

### Adhesion Assay

Jurkat T-cell adhesion to ICAM-1-coated plates was performed as described previously (4). Recombinant ICAM-1 was produced as described previously (4). Cells were labeled with 5-carboxyfluorescein. 1.5 x 10⁵ cells were plated for 20 minutes before removal of non-adherent cells by serial washes. The percentage of adherent cells was quantified with a plate reader (Synergy HT, BioTek Instruments, Inc., Winooski, Vt.) reading emissions at 525 nm.

### Microscopy

Live cells were plated in 35-mm dishes containing a no. 0 glass coverslip over a 15-mm cutout (MatTek, Ashland, Mass). Cells were maintained at 37° C. using a PDMI-2 microincubator (Harvard Apparatus, Holliston, Mass.). Individual cells were imaged before and after addition of stimuli. Images were acquired with a Zeiss 510 inverted laser scanning confocal microscope (Carl Zeiss Microlmaging, Inc., Thornwood, N.Y.) and processed with Adobe Photoshop CS.

### Mice

The Institutional Ethics Committee of Tel Aviv University approved the study. BALB/c female mice at 8 to 10 weeks of age were used. Oral administration of FTS-A required doses of 50 and 100 mg/kg (10). The compound 2,4-dinitro-l-fluorobenzene (DNFB) and olive oil were obtained from Sigma-Aldrich (St. Louis, Mo.). Mice were sensitized on the shaved abdomen with 50 μl of 0.5% DNFB in a vehicle of 4:1 acetone:olive oil. Mice were ear challenged with 20 μl t of 0.2% DNFB in a vehicle of 4:1 acetone:olive oil after 5 days. A constant area of the ears was measured immediately before challenge and 24 hours later with an engineer's micrometer (Ozaki Mfg. Co., Itabashi, Tokyo). Ear swelling was expressed as the difference in ear thickness before and after the challenge (11).

### Results

### FTS Analogs Inhibit Rap1 in Jurkat T Cells

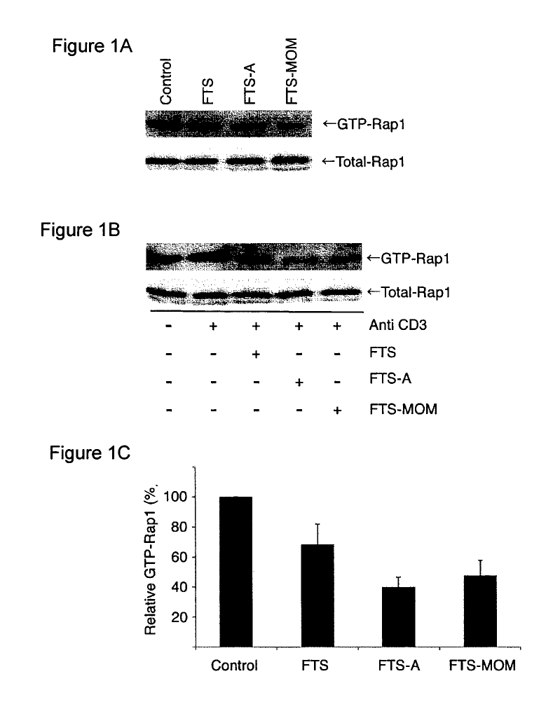

The experiments were designed to determine whether FTS and its analogs are able to inhibit GTP loading of Rap1. Quiescent Jurkat T cells were treated overnight with FTS, FTS-A, and FTS-MOM, each at a concentration of 50 μM. Cells were collected and the quantity of GTP.Rap1 was determined by pull-down assay. Compared with untreated cells (control), the amount of GTP.Rap1 decreased in all conditions (FIG. 1A). However, both FTS-A and FTS-MOM were superior to FTS in their ability to inhibit Rap1 activation (FIG. 1A). Since Rap1 is activated in T cells as a result of crosslinking the antigen receptor, the effect of FTS, and its analogs, on Rap1 activation in stimulated T cells, was studied. Cells were treated with FTS and its analogs overnight, and subsequently stimulated with anti CD3 antibodies for 10 minutes. As shown in FIG. 1B, all analogs were able to inhibit Rap1 activation in stimulated T cells. FTS lowered the amount of GTP.Rap1 by 32%, while FTS-A and FTS-MOM lowered it by 60% and 53%, respectively (FIG. 1C). Thus, FTS and its analogs are able to inhibit Rap1 activation in T cells stimulated through the antigen receptor. However, it appears that FTS-A and FTS-MOM were more potent Rap1 inhibitor compared to FTS.

### FTS Analogs Inhibit T Cell Adhesion

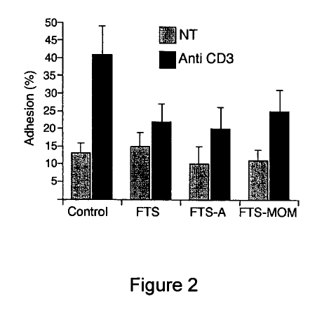

Since Rap1 is critical for T cell adhesion, the ability of FTS and its analogs, to inhibit adhesion of T cells to ICAM-1 coated plates was studied. T cells were treated overnight with FTS or its analogs. Cells were plated on ICAM-1 coated wells for 20 minutes followed by serial washings. A plate reader was used to evaluate the percentage of cells that remained in the wells. While 40% of stimulated T cells attached to the ICAM-1 coated wells, this number dropped by half in wells that were treated with the analogs (FIG. 2). No statistically significant difference could be found among the three agents. Thus, FTS, and its analogs, inhibit both Rap1 GTP loading and Rap1-dependent T cell adhesion.

### FTS-A Inhibition of Rap1 is Dose Dependent

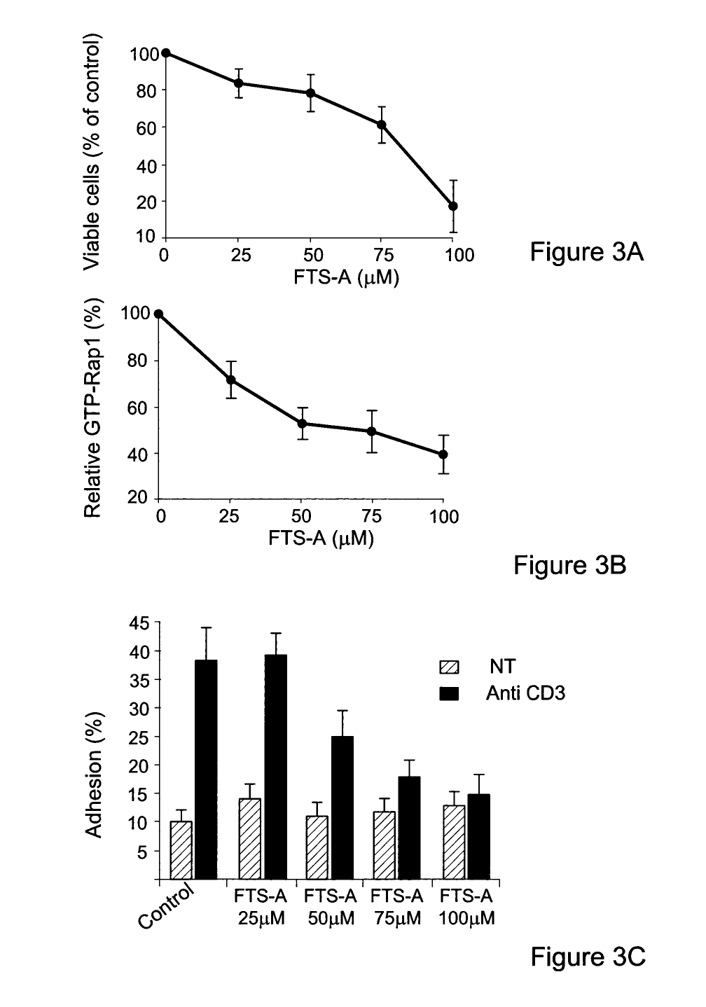

Out of the three agents (FTS, FTS-A and FTS-MOM), FTS-A showed the greatest amount of Rap1 inhibition. Thus, this analog was used for further experiments. First, I-cells were treated with various concentrations of FTS-A for 72 hours. As shown in FIG. 3A, with higher doses, the number of viable cells decreased, as assessed by tryptan blue exclusion. Notably, with higher doses of FTS-A, the amount of GTP.Rap1 further diminished indicating a dose-response relationship (FIG. 3B). Moreover, a similar dose-response relationship was found between FTS-A and the ability of the drug to inhibit adhesion to ICAM-1 coated plates (FIG. 3C).

### FTS-A Inhibits Rap1 Activation at the Plasma Membrane

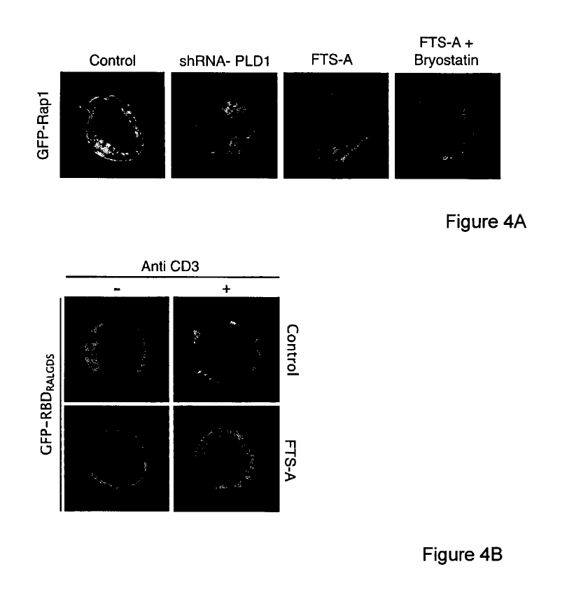

It has been reported that only the pool of Rap1 at the plasma membrane becomes GTP bound on lymphocyte activation (9). The following experiments were designed to study whether Rap1 inhibition by FTS-A is indeed restricted to that compartment. Jurkat T cells, expressing GFP tagged Rap1, were subjected to various agents (FIG. 4A), whereas the localization of Rap1 was recorded by confocal microscopy. As previously reported, knocking down phospholipase D1 (PLD1) resulted in inhibition of Rap1 and its association with the plasma membrane (FIG. 4A). FTS-A treatment for up to 72 hours did not change the localization of overexpressed Rap1 (FIG. 4A).

Rap1, like K-Ras, is associated with the plasma membrane through farnesylation that functions in conjunction with an adjacent polybasic sequence. Bryostatin-1, a protein kinase C (PKC) agonist, induced a rapid translocation of K-Ras from the plasma membrane to intracellular membranes (12). Nonetheless, the results showed that a combined treatment with Bryostatin-1 and FTS-A did not change the localization of Rap-1 (FIG. 4A). Thus, FTS-A does not change the bulk localization of Rap-1.

The effects of FTS are rather specific to the active GTP-bound forms of Ras proteins (7). To investigate the possible effect of FTS-A on localization of the active Rap1, the probe for activated Rap1, GFP-RBD_{RALGDS} (13), was utilized. Consistent with earlier reports (14), the results showed that when the cells were stimulated through the antigen receptor, the probe translocated to the plasma membrane, suggesting that Rap1 is activated at that compartment (FIG. 4B). In cells that were pretreated by FTS-A, the activation of Rap1 at the plasma membrane was blocked (FIG. 4B) although Rap1 remained associated with the plasma membrane (FIG. 4A). Thus, the pool of Rap1 that is inhibited by FTS-A is found at the plasma membrane.

### FTS-A Inhibits Contact Sensitivity Reaction in Animal Model

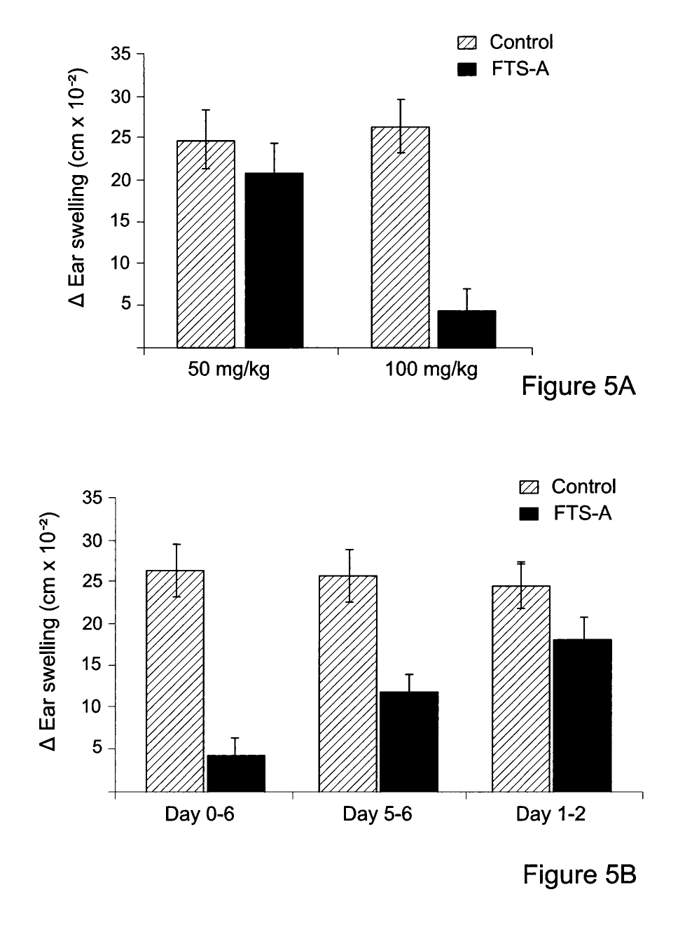

Next, the ability of FTS-A to inhibit Rap-1-dependent T cell adhesion in vivo was investigated by using contact sensitivity (15) as a model system. Animals were treated orally with two different concentrations of FTS-A (50 mg/kg and 100 mg/kg). As shown in FIG. 5A, only the higher concentration of FTS-A was able to inhibit ear swelling. When treatment was introduced only during the challenge phase (days 5-6), ear swelling was also attenuated. Treatment during the sensitization phase (days 0-2) did not prevent ear swelling (FIG. 5B), suggesting that FTS-A primarily blocked lymphocyte recruitment to the site of foreign antigen encounter.

Rap1 Inhibition by FTS-A is not Associated with Ras Inhibition

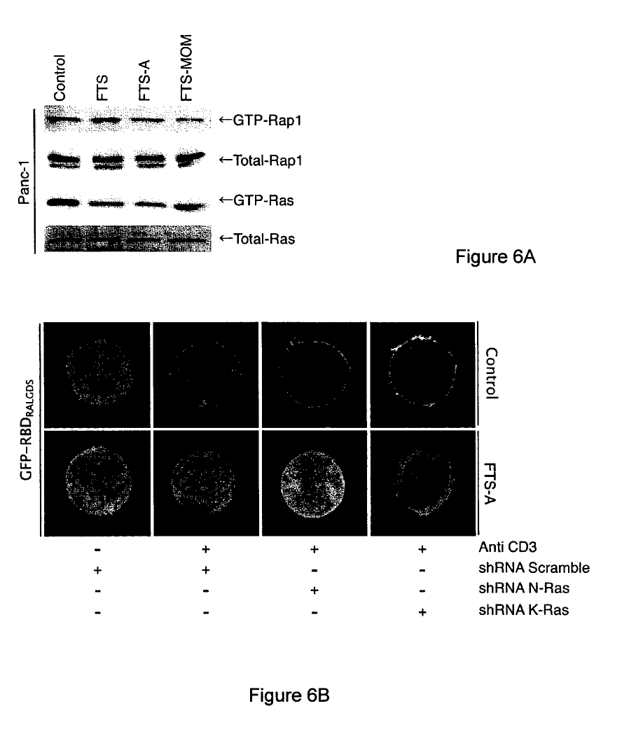

Since FTS is known to inhibit Ras, experiments were designed to determine whether the mechanism of Rap1 inhibition by FTS analogs is indirect and mediated by Ras inhibition. Applicants compared the inhibitory profile of FTS and its analogs on both Ras GTP loading and Rap1 GTP loading in Panc-1 cells (FIG. 6B). As previously reported, FTS-MOM is a weak Ras inhibitor, while FTS was comparable to FTS-A [Goldberg L, Haklai R, Bauer V, Heiss A, Kloog Y. New Derivatives of Farnesylthiosalicylic acid (Salirasib) for cancer treatment: Farnesylthiosalicylamide inhibits tumor growth in nude mice models. J Med Chem 2009; 52:197-205]. In contrast, the present results showed that FTS-MOM was by far the stronger Rap1 inhibitor, while the inhibitory effect of FTS on Rap1 was relatively modest (FIG. 6A). Thus, the extent of Ras and Rap1 inhibition by FTS and its analogs is not identical.

Furthermore, when both N-Ras and K-Ras were knocked down in Jurkat T cells via shRNA, the inhibitory effect of FTS-A on Rap1 was not interrupted, suggesting again that Rap1 inhibition is not Ras dependent (FIG. 6B).

### CONCLUSION

Applicants have demonstrated, surprisingly, that Rap1 activation is inhibited by FTS and its analogs. Applicants have also found that FTS-A exhibits much greater inhibitory activity toward Rap1 than FTS (FIG. 6A and 6B), suggesting that the effect of FTS-A on contact sensitivity (FIG. 5A) is through the inhibition of Rap1. Applicants have further shown that Rap1-mediated adhesion of lymphocytes was blocked by FTS-A in a dose-dependent manner.

Since there is considerable cross-talk between small G proteins, the question is raised as to whether the inhibitory effect of FTS-A on Rap1 is indeed direct or alternatively mediated through linkage to Ras. The present results present evidence that does not support such a Ras-Rap1 linkage. The effect of FTS-A on Rap1 activation was unchanged when Ras was knocked down, suggesting that Ras was not required for both Rap1 activation and for FTS-A-mediated inhibition of Rap1 activation (FIG. 6B).

Similar results were obtained in experiments with dominant negative Ras (data not shown). FTS-MOM was a strong Rap1 inhibitor (FIG. 6A) while it had only a minor inhibitory effect on Ras (FIG. 6B) (16). These results demonstrate a distinct profile of inhibition for these two small G binding proteins: relatively high selectivity of FTS towards Ras and of FTS-MOM toward Rap1.

Although FTS-A inhibited both Ras and Rap1 and FTS-MOM was more selective towards Rap1, applicants chose to use FTS-A for the contact sensitivity model system. Applicants did so because FTS-A showed the highest anti-Rap1 activity. Interestingly, even though Rap1 is attached to the cell membrane by a geranylgeranyl moiety, the FTS geranylgeranyl analogue (GGTS) did not prove to be a stronger Rap1 inhibitor (data not shown) .Rap1 is the key player in the development of delayed cutaneous hypersensitivity syndrome (2)] and it is likely that FTS-A affects mostly Rap1 under these circumstances. The results showed that the development of contact sensitivity reaction to DNFB in animal models was strongly inhibited by FTS-A. Contact sensitivity to DNFB has been extensively studied in mice. It requires both effective immune sensitization following cutaneous exposure to chemical haptens and antigen-specific elicitation (11, 17). The present results showed that treatment with FTS-A during the second exposure to the antigen was sufficient to block ear swelling. Therefore, FTS and its analogs such as FTS-A, collectively represented herein by formula (I), block the recruitment of the primed I-cells to the inflamed area, which demonstrates that they can be used to treat patients with cutaneous inflammatory diseases where T-cell adhesion and recruitment play a major role.

Citations of publications referenced herein:
1. Scheele J S, Marks R E, Boss G R. Signaling by small GTP in the immune system. Immunol Rev 2007; 218:92-101.
2. Mor A, Dustin M L, Philips M R. Small GTPases and LFA-1 reciprocally modulate adhesion and signaling. Immunol Rev 2007; 218:114-25
3. Dillon TJ, Carey K D, Wetzel S A, Parker D C, Stork P J S. Regulation of the small GTPase Rap1 and extracellular signal-regulated kinases by the costimulatory molecule CTLA-4. Mol Cell Biol 2005; 25:4117-28.
4. Mor A, Wynne J P, Ahearn I M, Dustin M L, Du G, Philips M R. Phospholipase D1 regulates lymphocyte adhesion via upregulation of Rap1 at the plasma membrane. Mol Cell Biol 2009; 29:3297-306.
5. Abram C L, Lowell C A. Leukocyte adhesion deficiency syndrome: a controversy solved. Immunol Cell Biol 2009; 87:440-2.
6. Kafri M, Kloog Y, Korczyn A D, Ferdman-Aronovich R, Drory V, Katzav A, Wirguin I, Chapman J. Inhibition of Ras attenuates the course of experimental autoimmune neuritis. Journal of Neuroimmunology 2005; 168:46-55.
7. Marom M, Haklai R, Ben-Baruch G, Marciano D, Egozi Y, Kloog Y. Selective inhibition of Ras-dependent cell growth by farnesylthiosalisylic acid. J Biol Chem 1995; 270:22263-70.
8. Haklai R. Orally administered FTS (salirasib) inhibits human pancreatic tumor growth in nude mice. Cancer Chemother Pharmacol 2008; 61:89-96.
9. Mor A, Campi G, Du G, Zheng Y, Foster D A, Dustin M L, Philips M R. The lymphocyte function-associated antigen-1 receptor costimulates plasma membrane Ras via phospholipase D2. Nat Cell Biol. 2007; 9:713-9.
10. Barkan B, Starinsky S, Friedman E, Stein R, Kloog Y. The Ras inhibitor farnesylthiosalicylic acid as a potential therapy for neurofibromatosis type 1. Clin Cancer Res 2006; 12:5533-18.
11. Zimber C, Ben-Efraim S, Weiss D W. Differential inhibition of contact sensitivity by suppressor T cells and suppressor factor induced by combined treatment with dinitrobenzenesulphonate and dinitrofluorobenzene. Immunology 1982; 45:449
12. Bivona T G, Quatela S E, Bodemann B O, Ahearn I M, Soskis M J, Mor A, Miura J, Wiener H H, Wright L, Saba SG, Yim D, Fein A, Perez de Castro I, Li C, Thompson C B, Cox A D, Philips M R. PKC regulates a farnesyl-electrostatic switch on K-Ras that promotes its association with Bcl-XL on mitochondria and induces apoptosis. Mol Cell 2006; 21:481-93.
13. Bivona T G, Philips M R. Analysis of Ras and Rap activation in living cells using fluorescent Ras binding domains. Methods 2005; 37:138-45.
14. Bivona T G, Wiener H H, Ahearn I M, Silletti J, Chiu V K, Philips M R Rap1 up-regulation and activation on plasma membrane regulates T cell adhesion. J Cell Biol. 2004; 164:461-70.
15. Claman Hn, Jaffee B D. Desensitization of contact allergy to DNFB in mice. J Immunology 1983; 131:2682-6.
16. Goldberg L, Haklai R, Bauer V, Heiss A, Kloog Y. New Derivatives of Farnesylthiosalicylic acid (Salirasib) for cancer treatment: Farnesylthiosalicylamide inhibits tumor growth in nude mice models. J Med Chem 2009; 52:197-205.
17. Bryce P J, Miller M L, Miyajima I, Tsai M, Galli S J, Oettgen H C. Immune sensitization in the skin is enhanced by antigen-independent effects of IgE. Immunity 2004; 20:381-92.

All patent publications and non-patent publications are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1-18. (canceled)

19. A method of treating a mammalian subject afflicted with a delayed cutaneous hypersensitivity condition, comprising administering to the subject a pharmaceutical composition comprising an effective amount of S-farnesylthiosalicylic acid (FTS) or a structural analog thereof, collectively defined in accordance with formula (I): 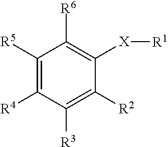
wherein X represents S; R¹ represents farnesyl, or geranyl-geranyl; R² is COOR⁷, CONR⁷R⁸, or COOCHR⁹R¹⁰, wherein R⁷ and R⁸ are each independently hydrogen, alkyl, or alkenyl; wherein R⁹ represents H or alkyl; and wherein R¹⁰ represents alkyl; and
wherein R³, R⁴, R⁵ and R⁶ are each independently hydrogen, alkyl, alkenyl, alkoxy, halo, trifluoromethyl, trifluoromethoxy, or alkylmercapto, and a pharmaceutically acceptable carrier.

20. The method according to claim 19, wherein the mammalian subject is a human.

21. The method according to claim 19, wherein the delayed cutaneous hypersensitivity condition is contact dermatitis.

22. The method according to claim 19, wherein the delayed cutaneous hypersensitivity condition is selected from the group consisting of allergic contact dermatitis, atopic dermatitis or irritant dermatitis.

23. The method according to claim 19, wherein the structural analog is FTS-amide wherein R¹ represents farnesyl, R² represents CONR⁷R⁸, and R⁷ and R⁸ both represent hydrogen.

24. The method according to claim 19, wherein the structural analog is FTS-methylamide wherein R¹ represents farnesyl, R² represents CONR⁷R⁸, and R⁷ represents hydrogen and R⁸ represents methyl.

25. The method according to claim 19, wherein the structural analog is FTS-dimethylamide wherein R¹ represents farnesyl, R² represents CONR⁷R⁸, and R⁷ and R⁸ each represents methyl.

26. The method according to claim 19, wherein FTS or its structural analog is administered orally, parenterally or transdermally.

27. The method according to claim 19 wherein FTS or its structural analog is administered in combination with another active agent for treating delayed cutaneous hypersensitivity condition.

28-29. (canceled)

30. The method according to claim 19, wherein the composition comprises an effective amount of FTS.
